(19) United States
(12) Patent Application Publication
(10) Pub. No.: US 2020/0113920 A1
(43) Pub. Date: Apr. 16, 2020

(54) COMPOSITIONS AND METHODS FOR TREATMENT OF MICROSPORIDIA INFECTION USING PROTEASOME INHIBITORS INCLUDING IXAZOMIB

(71) Applicant: BARNARD COLLEGE, New York, NY (US)

(72) Inventor: Jonathan Whitehead Snow, New York, NY (US)

(21) Appl. No.: 16/597,836

(22) Filed: Oct. 9, 2019

Related U.S. Application Data
(60) Provisional application No. 62/743,642, filed on Oct. 10, 2018.

(51) Int. Cl. A61K 31/69; A61K 31/4965; A61K 31/5377; A61K 31/407; A61K 31/426; A61K 31/336; A61K 38/06; A61P 31/10; A61F 7/00; G01N 33/50; C12Q 1/686; G01N 15/14

(52) U.S. Cl. CPC A61K 31/69; A61K 31/4965; A61K 31/5377; A61K 31/407; A61K 31/426; G01N 15/1429; A61K 38/06; A61P 31/10; A61F 7/00; G01N 33/5091; C12Q 1/686; A61K 31/336

(57) ABSTRACT

The present disclosure provides, inter alia, methods for treating infectious diseases, e.g., a microsporidia infection caused by Nosema ceranae, in a subject such as, e.g., a honeybee, using proteasome inhibitors including Ixazomib. Methods for monitoring the progress of a microsporidia infection in a subject, and methods for measuring the intensity of a microsporidia infection in a subject, are also provided.

### CROSS-REFERENCE TO RELATED APPLICATION

The present application claims benefit to U.S. Provisional Application Ser. No. 62/743,642, filed Oct. 10, 2018. The entire content of the above application is incorporated by reference as if recited in full herein.

### FIELD OF DISCLOSURE

The present disclosure provides, inter alia, methods for treating infectious diseases in a subject using proteasome inhibitors.

### BACKGROUND OF THE DISCLOSURE

The Western Honey Bee, Apis mellifera, provides pollination services of critical importance to humans in both agricultural and ecological settings (Potts et al. 2010). Honey bee colonies have suffered from increased mortality in recent years that is likely caused by a complex set of interacting stresses (Coulson et al. 2015). Among the environmental stressors implicated in honey bee disease, there has been intensifying focus on the role of microbial attack on honey bee health (Evans et al. 2011). The microsporidian species Nosema ceranae and Nosema apis represent one group of microbes that can cause individual mortality in honey bees, and have been implicated in colony collapse (Higes et al. 2013; Fries 2010; Fries 2014). Nosema ceranae was first identified in the late 1990's in the Asian honey bee (Apis cerana) (Fries et al. 1996) and was not observed in Apis mellifera colonies until the mid 2000's (Higes et al. 2006; Huang et al. 2007; Chen and Huang 2010). As N. ceranae has become highly prevalent in managed colonies of European honey bees in a timeframe concomitant with colony losses (Cox-Foster et al. 2007; Klee et al. 2007), it has received more attention. N. ceranae spores infect the midgut of honey bees, causing energetic stress, epithelial damage, and when untreated, death (Cornman et al. 2012; Mayack and Naug 2009; Dainat et al. 2012; Dussaubat et al. 2012; Higes et al. 2007; Higes et al. 2013). In addition, infection has been associated with a number of physiological and behavioral changes that likely affect individual contribution to the colony (Fries 2010; Goblirsch et al. 2013; Alaux et al. 2012; Lach et al. 2015). Gene expression analysis of the infected bees has uncovered a variety of transcriptional changes associated with infection, most notably alterations in epithelial regeneration and metabolic processes (Fries 1993; Holt et al. 2013; Aufauvre et al. 2014).

N. ceranae infection can currently be controlled by treatment with the drug Fumagillin (Higes et al. 2006; Huang et al. 2007; Chen and Huang 2010; Higes et al. 2011), a methionine aminopeptidase 2 inhibitor. However, high doses of this drug are toxic to all eukaryotic cells and evidence suggests that N. ceranae can evade suppression in some circumstances (Huang et al. 2013). Even more critically, this drug is slated to become unavailable in the near future due to production problems. Thus, efforts to find alternative treatment strategies are critical to protect susceptible organisms, e.g., honey bees, from this parasite. The search for novel treatments has been extensive, but has not yet resulted in effective alternatives (Holt and Grozinger 2016).

### SUMMARY OF THE DISCLOSURE

Microsporidia are obligate intracellular parasites that that cause widespread infections in nature, but are relatively understudied compared to microbial pathogens representing other taxonomic groups, such as bacteria and non-microsporidial fungi. These pathogens infect diverse invertebrate species that play important roles throughout our food production system as well as causing disease in immunocompromised humans.

Pharmacological inhibition of the proteasome has been widely used in the treatment of protozoan parasites and beyond. It is hypothesized that this approach might work to control microsporidia infection as well. Based on available microsporidia genomes in the KEGG database (N. ceranae, Encephalitozoon hellem, Encephalitozoon cuniculi, Encephalitozoon intestinalis, Encephalitozoon romalea), microsporidia are missing 3 of the 13 genes encoding Regulatory particle of non-ATPase (Rpn) (Rpn12, Rpn13, and Rpn 15) (FIG. 1). These absences suggest potentially altered function or regulation of the proteasome in microsporidia. Closer examination of N. ceranae UPS genes, using Saccharomyces cerevisiae for comparison (Finley et al. 2012), it was found that this species, and the closely related Nosema apis, are also lacking two of the seven α gating subunits and one of the seven β catalytic subunits found in the 20S core particle in all other eukaryotes studied to date (FIG. 1).

Recent studies have shown that Fumagillin does not fully control N. ceranae infection in the field, making the discovery of novel anti-microsporidian agents with activity against this species of paramount important. During N ceranae infection, feeding bees either of two proteasome inhibitors (Ixazomib (1st oral inhibitor) and MG132) resulted in a dramatic reduction in infection intensity. While MG132 was not as efficacious as Fumagillin, Ixazomib had better efficacy than Fumagillin. Thus, proteasome inhibition offers a new treatment strategy for honey bee infection by Nosema spp. and may help in targeting microsporidian infections affecting diverse host species.

Accordingly, one embodiment of the present disclosure is a method for treating an infectious disease in a subject in need thereof, comprising administering an effective amount of a proteasome inhibitor to the subject.

Another embodiment of the present disclosure is a method for monitoring the progress of a microsporidia infection in a subject, comprising: (a) obtaining a cell sample from the subject; (b) staining the cell sample with one or more cell dyes; (c) identifying distinct cell populations in the sample based on the dye signals; (d) determining the infection stage of the subject by comparing the result of step (c) with that of an uninfected subject; and (e) initiating a treatment protocol for the subject based on the infection stage determined in step (d).

A further embodiment of the present disclosure is a method for measuring the intensity of a microsporidia infection in a subject, comprising: (a) obtaining a cell sample from the subject; (b) measuring the infection intensity by determining the relative amount of the microsporidia DNA versus host DNA of the subject; and (c) initiating a treatment protocol for the subject based on the infection intensity measured in step (b).

The present disclosure further provides a method for treating a Nosema ceranae infection in a susceptible organism, such as, e.g., a honeybee, comprising administering an effective amount of Ixazomib to the susceptible organism, such as, e.g., the honeybee.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1 is a schematic of the structure of the N. ceranae proteasome. The 19S (or PA700) proteasome complex, known as the Regulatory Particle (RP), is made up of two subcomplexes, the lid and the base. The 19S RP contains ˜20 subunits that belongs to either the Regulatory particle of triple-ATPase (Rpt) group or the Regulatory particle of non-ATPase (Rpn) (Bard et al. 2018).

FIG. 2A shows that MG132 reduces N. ceranae infection intensity. *p<0.05.

FIG. 2B shows that Ixazomib reduces N. ceranae infection intensity by both spore counting (left panel) and DNA analysis (right panel). **p<0.01.

FIG. 3 shows that proteasome inhibitors reduce N. ceranae infection intensity by both spore counting (left panels) and DNA analysis (right panels). a≠b≠c≠d with p<0.05.

FIGS. 4A-4F show that Ixazomib stably reduces N. ceranae infection level in a dose and time dependent manner. a≠b≠c with p<0.05. FIGS. 4A, 4C and 4E provide results of spore counting. FIGS. 4B, 4D and 4F provide results of DNA analysis.

FIG. 5 shows that Ixazomib and Ixazomib citrate do not reduce honey bee survival at doses 4-fold above those that are effective at reducing N. ceranae infection intensity.

FIG. 6 shows that treatment with 10-100 nM Ixazomib reduces E. hellem infection intensity in a four-day infection of rabbit kidney cell line (RK-13) cells (left panel) without substantial reduction in rabbit cell numbers (right panel).

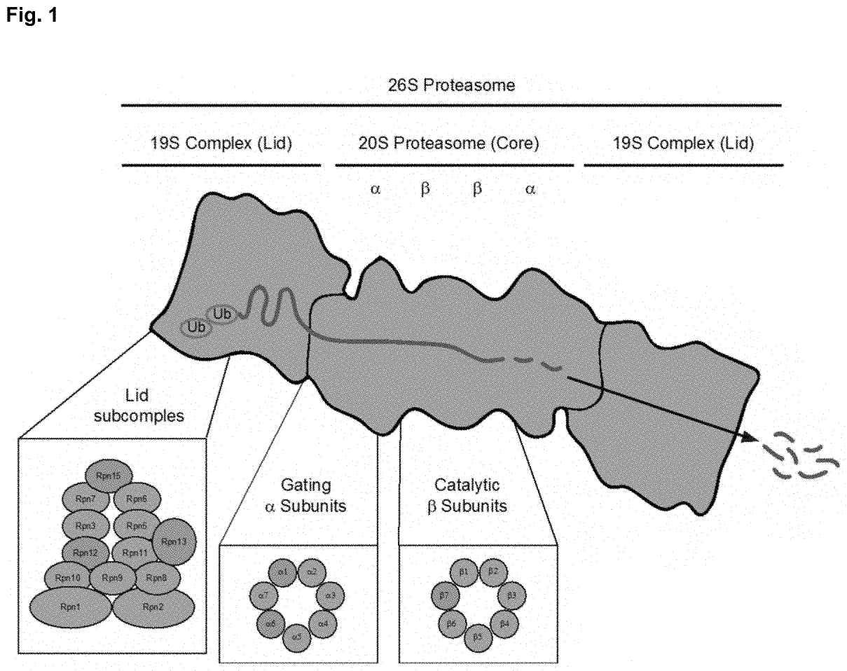

### DETAILED DESCRIPTION OF THE DISCLOSURE

The present disclosure explores new approaches to treat microsporidia infection in various susceptible organisms referred to interchangeably herein also as “host subjects” or “subjects”, such as, e.g., honeybee. The present disclosure also provides results of proteasome inhibition by testing lead compounds in field trials.

Accordingly, one embodiment of the present disclosure is a method for treating an infectious disease in a subject in need thereof, comprising administering an effective amount of a proteasome inhibitor to the subject.

As used herein, an “infectious disease” refers to a disorder caused by microorganisms (also collectively called pathogens)—such as bacteria, viruses, fungi or parasites. Signs and symptoms vary depending on the microorganism causing the infection, but often include fever, fatigue, inflammation and others. In some embodiments, the infectious disease is a microsporidia infection caused by, e.g., Nosema ceranae or Encephalitozoon hellem.

In some embodiments, the subject is selected from the group consisting of insects, fish, birds and mammals. Preferably, the subject is a bee, including a honeybee such as, e.g., A. mellifera. As used herein, a “honey bee” means a bee capable of producing honey, including wild honey-producing bees and those honey-producing bees which are cultivated for their ability to produce honey on a commercial scale.

As used herein, “proteasomes” are protein complexes which degrade unneeded or damaged proteins by proteolysis, a chemical reaction that breaks peptide bonds. Enzymes that help such reactions are called proteases. Proteasomes are found inside all eukaryotes and archaea, and in some bacteria. In eukaryotes, proteasomes are located in the nucleus and the cytoplasm. Proteasomes are part of a major mechanism by which cells regulate the concentration of particular proteins and degrade misfolded proteins via ubiquitination and proteasomal degradation (i.e., the ubiquitin-proteasome system). As used herein, a “proteasome inhibitor” refers to an agent that blocks the action of proteasomes. Non-limiting examples of proteasome inhibitors include Bortezomib, Carfilzomib, Marizomib, Ixazomib, ixazomib citrate, Oprozomib, Delanzomib, MG132 (both S and R stereoisomers), Dexazomib, Epoxomicin, HMB-Val-Ser-Leu-VE, MG-262 and combinations thereof. In some embodiments, the proteasome inhibitor is Ixazomib.

In some embodiments, the method disclosed above further comprises applying heat-shock to the subject.

Another embodiment of the present disclosure is a method for monitoring the progress of a microsporidia infection in a subject, comprising: (a) obtaining a cell sample from the subject; (b) staining the cell sample with one or more cell dyes; (c) identifying distinct cell populations in the sample based on the dye signals; (d) determining the infection stage of the subject by comparing the result of step (c) with that of an uninfected subject; and (e) initiating a treatment protocol for the subject based on the infection stage determined in step (d). In some embodiments, the microsporidia infection is caused by Nosema ceranae. In some embodiments, the subject is an insect, such as a bee, particularly a honeybee. In some embodiments, the one or more cell dyes are selected from a chitin-binding dye, a lysosome dye, and combinations thereof. In certain embodiments, step (c) of the method disclosed above is carried out by flow cytometry.

A further embodiment of the present disclosure is a method for measuring the intensity of a microsporidia infection in a subject, comprising: (a) obtaining a cell sample from the subject; (b) measuring the infection intensity by determining the relative amount of the microsporidia DNA versus host DNA of the subject; and (c) initiating a treatment protocol for the subject based on the infection intensity measured in step (b). In some embodiments, the microsporidia infection is caused by Nosema ceranae or Encephalitozoon hellem. In some embodiments, the subject is an insect, such as a bee, particularly a honeybee. In certain embodiments, step (b) of the method disclosed above is carried out by quantitative PCR.

In certain embodiments disclosed herein, a “treatment protocol” is carried out. As used herein, a “treatment protocol” includes all of the methods disclosed herein, particularly treating a subject in need thereof with any of the proteasome inhibitors disclosed herein, including, e.g., Ixazomib and/or Ixazomib citrate.

The present disclosure further provides a method for treating a Nosema ceranae infection in a susceptible organism, such as, e.g., a honeybee, comprising administering an effective amount of Ixazomib to the susceptible organism, such as, e.g., the honeybee. In certain embodiments, this method further comprises placing the susceptible organism, such as, e.g., the honeybee in an environment with an elevated temperature of 45° C. for a sufficient time to treat the infection.

### EXAMPLES

The following examples are provided to further illustrate certain aspects of the present disclosure. These examples are illustrative only and are not intended to limit the scope of the disclosure in any way.

### Example 1

### Proteasome Inhibition Controls Existing Infections by N. ceranae in Experimentally and Naturally Infected Bees

The goal was to determine how proteasome inhibition would impact N. ceranae infection in honey bees. MG132 is a commercially available (e.g., from Cayman Chemical) synthetic peptide aldehyde proteasome inhibitor having the structure of

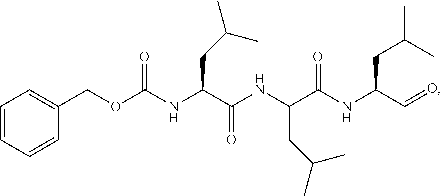 [SMILES: CC(C)CC(NC(=O)[C@H](CC(C)C)NC(=O)OCc1ccccc1)C(=O)N[C@H](C=O)CC(C)C]

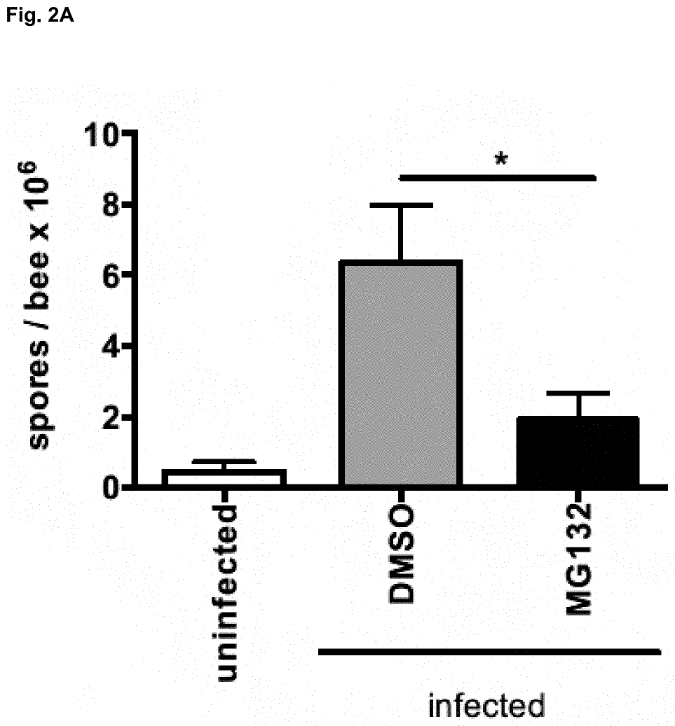

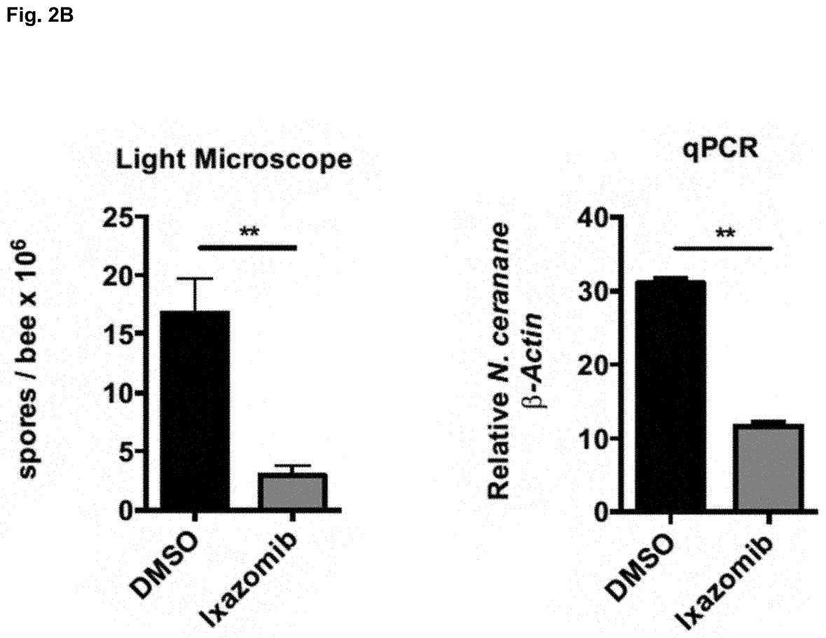

that works by reacting with the β5 subunit responsible for chymotrypsin-like activity (Goldberg, 2012). After experimentally infecting bees, infected bees were fed sucrose syrup or sucrose syrup containing 500 μM MG132 for 2 days starting on day 6 post infection. On day 8 post infection, spore levels were then measured using light microscopy to determine the effects of proteasome inhibition on N. ceranae infection intensity. It was found that feeding infected bees MG132 for 48 hours resulted in a dramatic reduction in infection intensity in infected bees (FIG. 2A). These results were similar to that observed over two days of feeding the canonical anti-Nosema agent Fumagillin (data not shown). It was also found similar results for bees captured on the landing board of a heavily infected colony (data not shown). MG132 is a poorly bioavailable proteasome inhibitor with known toxicity in multicellular eukaryotes. However, with these results, testing of other next generation proteasome inhibitors was warranted. Significant effort has been directed towards finding novel strategies for targeting the proteasome (Goldberg, 2012; Śledź and Baumeister, 2016; Cromm and Crews, 2017), providing a number of pharmacologic agents with better efficacy, improved bioavailability, and increased selectivity. We first tested the ability of ixazomib, a modified peptide boronic acid that binds the β5 site of the 20 S proteasome and inhibits proteasome activity (Kupperman et al. 2010), to reduce N. ceranae infection in honey bees. Ixazomib is commercially available (e.g., from Selleck Chemincals) and has the structure of

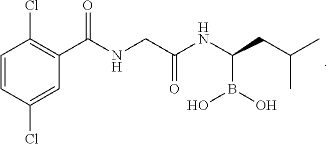 [SMILES: CC(C)C[C@H](NC(=O)CNC(=O)c1cc(Cl)ccc1Cl)B(O)O]

Using bees from a highly infected colony (prevalence>90% infected), we fed bees Ixazomib or a DMSO vehicle control for 2 days and observed a striking reduction in N. ceranae infection by both spore-counting and DNA analysis (FIG. 2B).

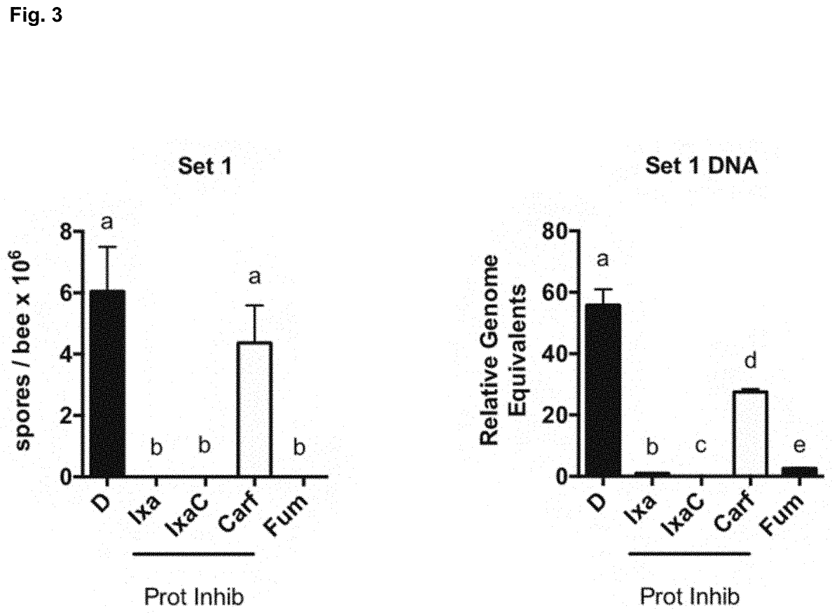

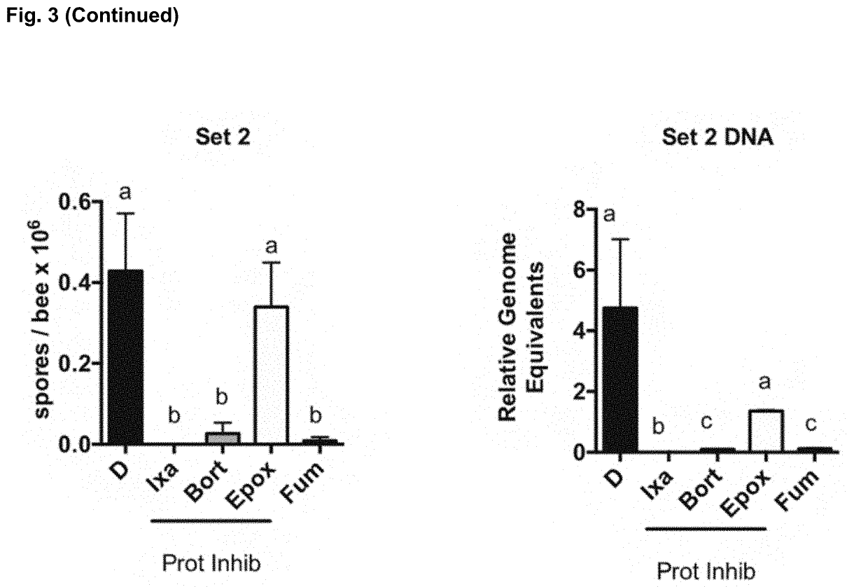

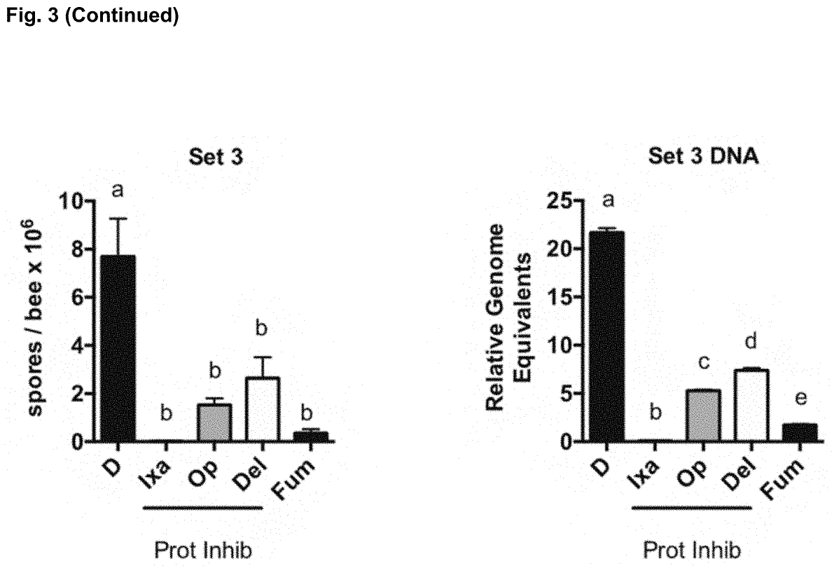

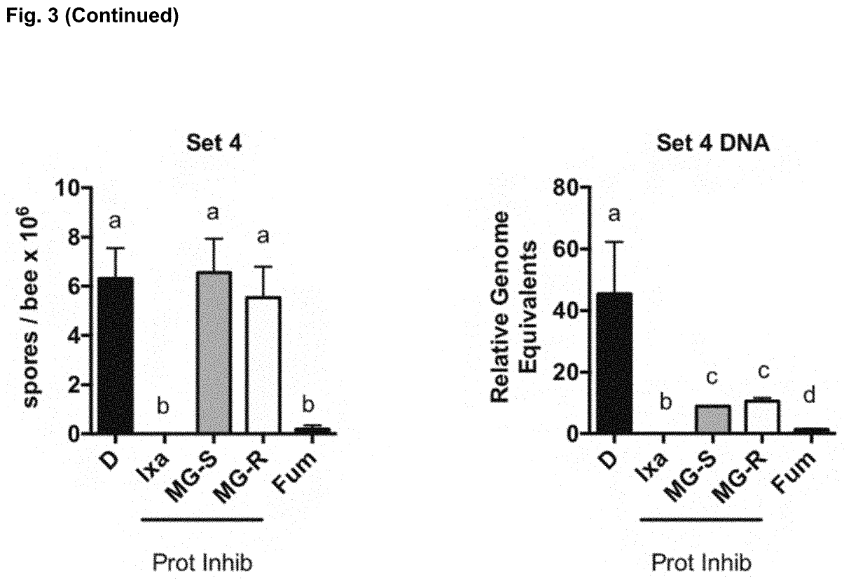

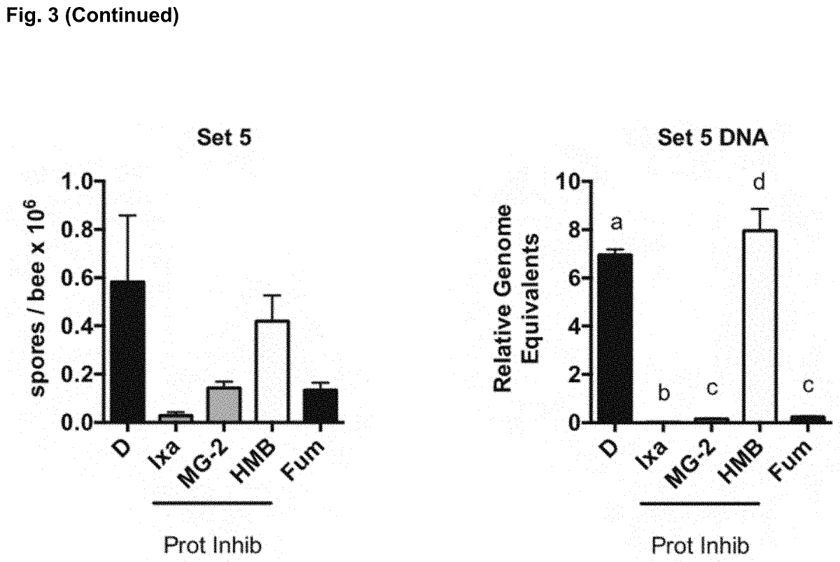

These results justified further testing of ixazomib and other proteasome inhibitors as possible anti-Nosema agents. To standardize experiments by using age-matched bees and to allow for longer treatment periods, we used newly enclosed bees and tested the effects of a number of commercially available proteasome inhibitors, including Ixazomib (MLN2238) (purchased from Selleck Chemicals), Ixazomib citrate (MLN9708) (purchased from Selleck Chemicals), Oprozomib (ONX 0912) (purchased from Selleck Chemicals), Dexazomib (purchased from Selleck Chemicals), Carfilzomib (purchased from Selleck Chemicals), Bortezomib (purchased from Selleck Chemicals), Epoxomicin (purchased from Selleck Chemicals), HMB-Val-Ser-Leu-VE (purchased from Cayman Chemical), MG-262 (purchased from APExBIO), and the two stereoisomers of MG132 (S and R) (purchased from Cayman Chemical) as well as Fumagillin (purchased from Selleck Chemicals). On day two post-eclosion, N. ceranae spores (5×10⁶ ml) were fed to bees in sucrose solution ad libitum (Fries et al. 2013) for 48 hours. At 3 days post infection, honey bees in individual cages were fed sucrose solution containing one of the pharmacologic agents at 40 μM or vehicle control alone. For each trial, we tested pairs of novel compounds simultaneously with an untreated group, a Fumagillin treated group, and an Ixazomib-treated group. After 4 days of drug feeding, honey bee midguts were dissected, and infection levels were assessed by spore counting and quantitative PCR. We observed reductions in infection level by relative genome equivalents for all tested proteasome inhibitors except HMB-Val-Ser-Leu-VE (FIG. 3). There were varying levels of impact on N. ceranae infection with Ixazomib and its citrate salt being the most effective at reducing infection levels.

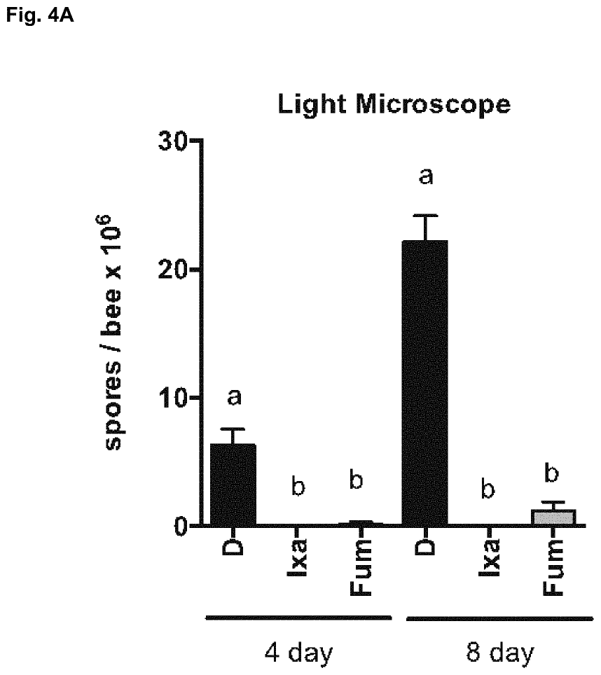

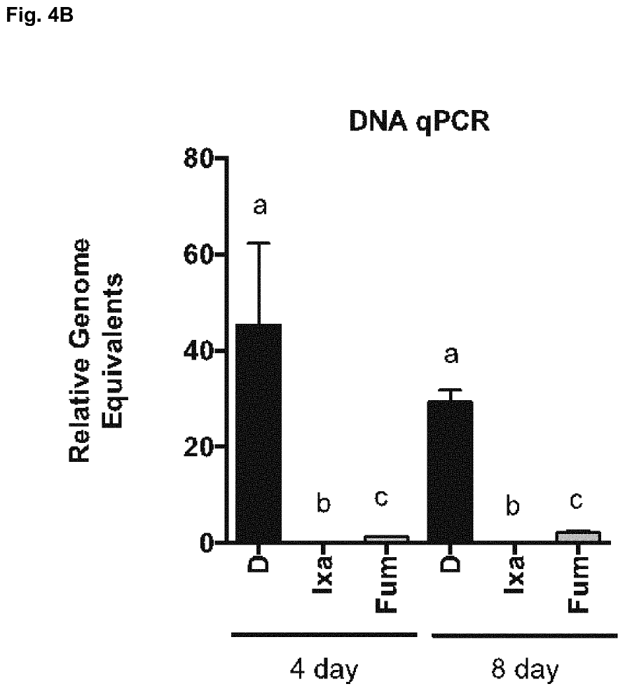

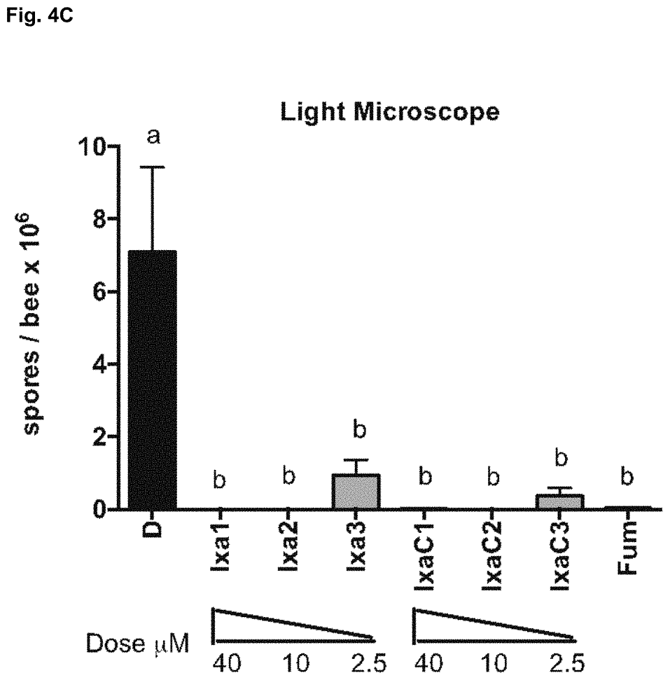

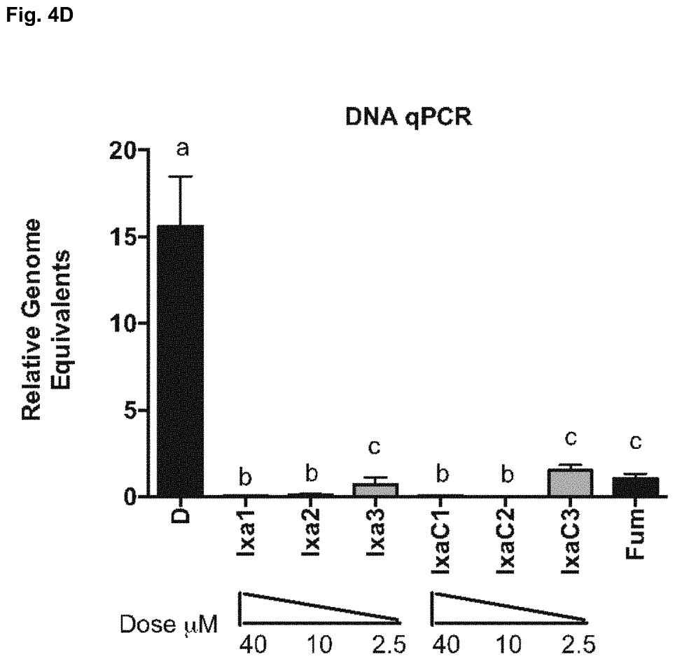

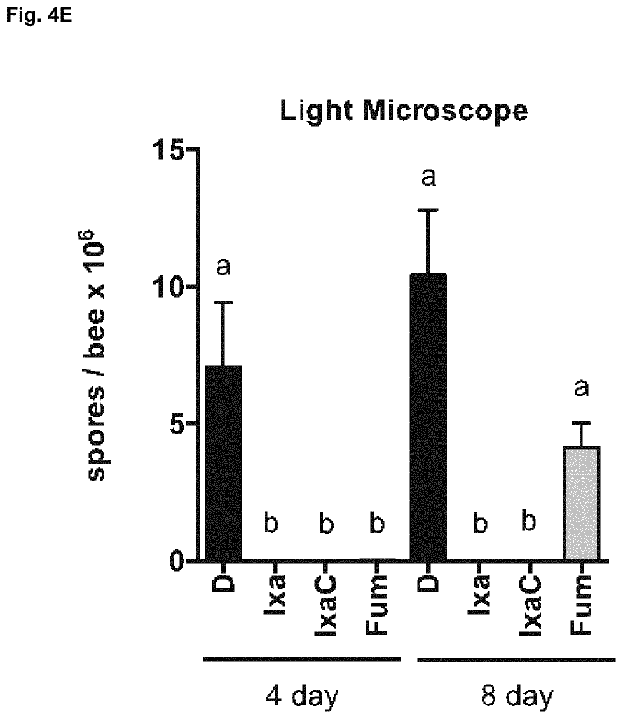

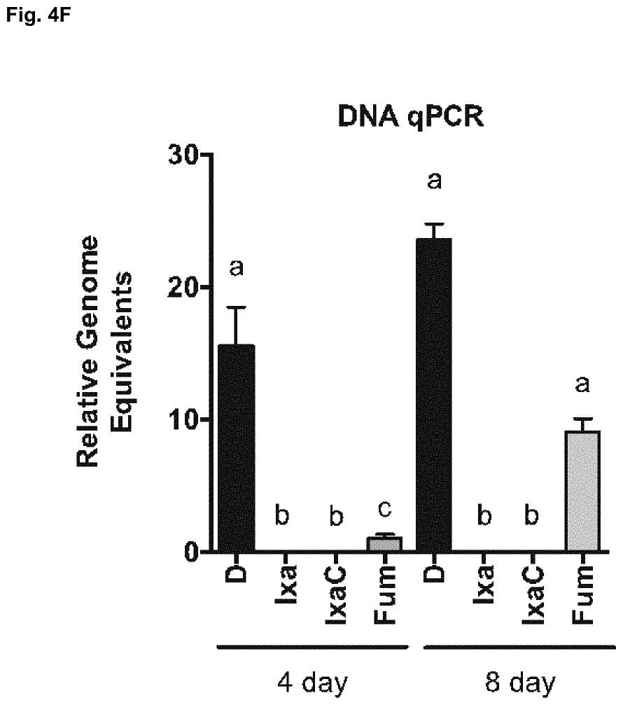

We focused on ixazomib and ixazomib citrate for further experiments. Again using newly eclosed bees, we treated infected bees for up to eight days with ixazomib, Fumagillin, or sucrose solution alone, and measured infection level by spore counting and DNA on days 4 and 8 post initiation of treatment (FIGS. 4A and 4B). We also looked at the dose responsiveness of ixazomib and ixazomib citrate on N. ceranae infection levels and observed a similar reduction in infection intensity at 10 μM and a diminished, but still robust, reduction in infection intensity at 2.5 μM of both ixazomib and ixazomib citrate by spore counting and DNA analysis (FIGS. 4C and 4D). Finally, the goal was to determine whether any rebound of N. ceranae infection was observed after cessation of treatment by ixazomib and ixazomib citrate as has been reported for Fumagillin (Huang et al. 2013). Infected newly eclosed bees were treated with sucrose solution containing ixazomib, ixazomib citrate, Fumagillin, or vehicle alone for four days. We then switched all cages to sucrose solution alone for 4 days and then measured infection level by spore counting and DNA. We observed that infection intensity stayed the same for bees receiving sucrose solution for the whole experiment, increased for those bees treated with Fumagillin first, and decreased for those bees fed either ixazomib or ixazomib citrate first (FIGS. 4E and 4F). Suggesting that even with a short treatment course, ixazomib and ixazomib citrate can eliminate infection with no danger of subsequent reemergence.

### Example 2

### Proteasome Inhibitors Ixazomib and Ixazomib Citrate are Well Tolerated by Bees at Doses Above Those Needed to Remove N. ceranae

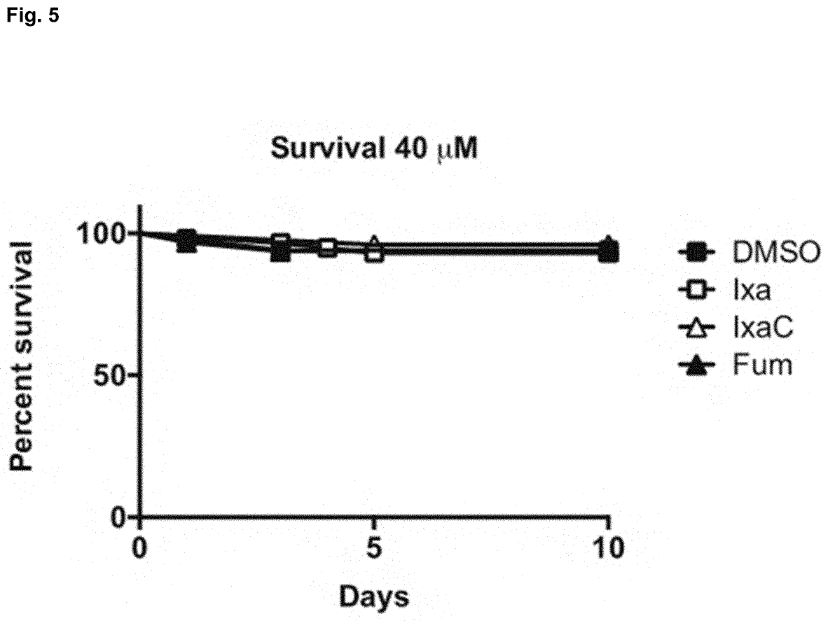

To assess the impact of ixazomib treatment on age-matched honey bees, newly emerged bees were fed sucrose solution containing ixazomib, ixazomib citrate, Fumagillin (all at 40 μM), or vehicle alone for 10 days starting on day 3 post-eclosion. We found very low mortality of bees in this experiment and no differences in the survival between treatments (FIG. 5).

### Example 3

### Proteasome Inhibition Impacts Microsporidia Infection of Other Host Species

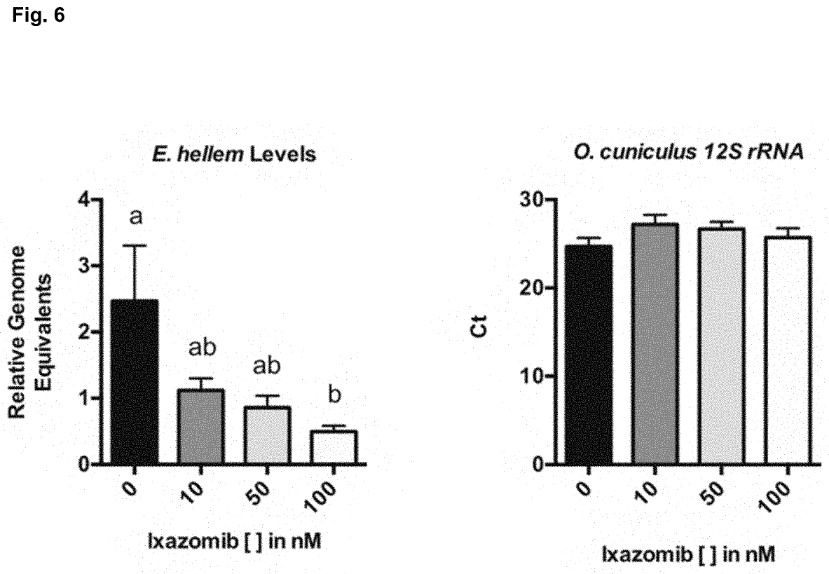

Examination of the genomes of other microsporidia suggested that other species in this group lack these same proteasome components. Thus, it was hypothesized that pharmacological inhibition of the UPS will reduce infection intensity of diverse microsporidia in their respective host species. To test this hypothesis, we chose to focus on Encephalitozoon hellem, a microsporidia species first described as the cause of keratoconjunctivitis in 3 AIDS patients in 1991 (Didier et al. 1991) and subsequently observed in numerous immunocompromised patients (Weiss and Becnel, 2014). The genome of E. hellem has been sequenced (Pombert et al. 2012) and it is easily culturable (Molestina et al. 2014), making it ideal for such studies. Indeed, preliminary results showed that treatment with 10-100 nM ixazomib reduces E. hellem infection intensity in a four-day infection of rabbit kidney cell line (RK-13) cells comparable to treatment with 50 ng/ml (Didier et al. 1991) Fumagillin as assessed by a qPCR-based assay of pathogen and host cell genome equivalents (FIG. 6) without substantial reduction in rabbit cell numbers.

### CITED DOCUMENTS

The following references, to the extent that they provide exemplary procedural or other details supplementary to those set forth herein, are specifically incorporated herein by reference.
1. Alaux, C. D., Kemper, N., Kretzschmar, A. & Le Conte, Y. Brain, physiological and behavioral modulation induced by immune stimulation in honeybees (Apis mellifera): A potential mediator of social immunity? Brain Behavior and Immunity 26, 1057-1060 (2012).
2. Aufauvre, J. et al. Transcriptome Analyses of the Honeybee Response to Nosema ceranae and Insecticides. PLoS ONE 9, e91686 (2014).
3. Bailey, L. The natural mechanism of suppression of Nosema apis Zander in enzootically infected colonies of the honey bee, Apis mellifera Linnaeus. Journal of insect pathology (1959).
4. Bard, J. A. M. et al. Structure and Function of the 26S Proteasome. Annu. Rev. Biochem. 87, 697-724 (2018).
5. Chen, Y. P. & Huang, Z. Y. Nosema ceranae, a newly identified pathogen of Apis mellifera in the USA and Asia. Apidologie 41, 364-374 (2010).
6. Cornman, R. S. et al. Pathogen webs in collapsing honey bee colonies. PLoS ONE 7, e43562 (2012).
7. Cox-Foster, D. L. et al. A metagenomic survey of microbes in honey bee colony collapse disorder. Science 318, 283-287 (2007).
8. Cromm P M, Crews C M. The Proteasome in Modern Drug Discovery: Second Life of a Highly Valuable Drug Target. ACS Cent Sci. 2017; 3: 830-838.
9. Dainat, B., Evans, J. D., Chen, Y. P., Gauthier, L. & Neumann, P. Predictive markers of honey bee colony collapse. PLoS ONE 7, e32151 (2012).
10. Delaplane, K. S., van der Steen, J. & Guzmán-Novoa, E. Standard methods for estimating strength parameters of Apis mellifera colonies. Journal of Apicultural Research. 52, (2013).
11. Didier E S et al. Isolation and characterization of a new human microsporidian, Encephalitozoon hellem (n. sp.), from three AIDS patients with keratoconjunctivitis. J Infect Dis. 1991 March; 163(3):617-21.
12. Dussaubat, C. et al. Gut Pathology and Responses to the Microsporidium Nosema ceranae in the Honey Bee Apis mellifera. PLoS ONE 7, e37017 (2012).
13. Erler, S., Lommatzsch, S. & Lattorff, H. M. G. Comparative analysis of detection limits and specificity of molecular diagnostic markers for three pathogens (Microsporidia, Nosema spp.) in the key pollinators Apis mellifera and Bombus terrestris. Parasitol Res 110, 1403-1410 (2011).
14. Evans, J. D. & Schwarz, R. S. Bees brought to their knees: microbes affecting honey bee health. Trends Microbiol 19, 614-620 (2011).
15. Fayer, R. & Duran, M. S. in Microsporidia: Pathogens of Opportunity (eds. Weiss, L. & Becnel, J. J.) (John Wiley and Sons, 2014).
16. Finley, D., Ulrich, H. D., Sommer, T. & Kaiser, P. The Ubiquitin-Proteasome System of Saccharomyces cerevisiae. Genetics 192, 319-360 (2012).
17. Fries, I. A parasite in the honey bee colony. Bee World 74, 5-19 (1993).
18. Fries, I. et al. Standard methods for Nosema research. Journal of Apicultural Research. 52, (2013).
19. FRIES, I. Microsporidia, Honeybees, and Colony Collapse Disorder. Microsporidia: Pathogens of Opportunity (2014).
20. Fries, I. Nosema ceranae in European honey bees (Apis mellifera). J Invertebr Pathol 103 Suppl 1, S73-9 (2010).
21. Fries, I., Feng, F., daSilva, A., Slemenda, S. B. & Pieniazek, N. J. Nosema ceranae n sp (Microspora, Nosematidae), morphological and molecular characterization of a microsporidian parasite of the Asian honey bee Apis cerana (Hymenoptera, Apidae). European Journal of Protistology 32, 356-365 (1996).
22. Gisder, S. & Genersch, E. Identification of Candidate Agents Active against N. ceranae Infection in Honey Bees: Establishment of a Medium Throughput Screening Assay Based on N. ceranae Infected Cultured Cells. PLoS ONE 10, e0117200 (2015).
23. Goblirsch, M., Huang, Z. Y. & Spivak, M. Physiological and Behavioral Changes in Honey Bees (Apis mellifera) Induced by Nosema ceranae Infection. PLoS ONE 8, e58165 (2013).
24. Goldberg, A. L. Development of proteasome inhibitors as research tools and cancer drugs. Journal of Cell Biology 199, 583-588 (2012).
25. Goulson, D., Nicholls, E., Botias, C. & Rotheray, E. L. Bee declines driven by combined stress from parasites, pesticides, and lack of flowers. Science 347, 1255957 (2015). 26. Higes, M. et al. Apoptosis in the pathogenesis of Nosema ceranae (Microsporidia: Nosematidae) in honey bees (Apis mellifera). Environmental Microbiology Reports 5, 530-536 (2013).
27. Higes, M. et al. The stability and effectiveness of fumagillin in controlling Nosema ceranae (Microsporidia) infection in honey bees (Apis mellifera) under laboratory and field conditions. Apidologie 42, 364-377 (2011).
28. Higes, M., García-Palencia, P., Martin-Hernández, R. & Meana, A. Experimental infection of Apis mellifera honeybees with Nosema ceranae (Microsporidia). J Invertebr Pathol 94, 211-217 (2007).
29. Higes, M., Martin, R. & Meana, A. Nosema ceranae, a new microsporidian parasite in honeybees in Europe. J Invertebr Pathol 92, 93-95 (2006).
30. Higes, M., Meana, A., Bartolomé, C., Botías, C. & Martin-Hernández, R. Nosema ceranae (Microsporidia), a controversial 21st century honey bee pathogen. Environmental Microbiology Reports 5, 17-29 (2013).
31. Holt, H. L. & Grozinger, C. M. Approaches and Challenges to Managing Nosema (Microspora: Nosematidae) Parasites in Honey Bee (Hymenoptera: Apidae) Colonies. Journal of Economic Entomology 109, 1487-1503 (2016).
32. Holt, H. L., Aronstein, K. A. & Grozinger, C. M. Chronic parasitization by Nosema microsporidia causes global expression changes in core nutritional, metabolic and behavioral pathways in honey bee workers (Apis mellifera). BMC Genomics 14, 799 (2013).
33. Huang, W.-F., Jiang, J.-H., Chen, Y.-W. & Wang, C.-H. A Nosema ceranae isolate from the honeybee Apis mellifera. Apidologie 38, 30-37 (2007).
34. Huang, W.-F., Solter, L. F., Yau, P. M. & Imai, B. S. Nosema ceranae Escapes Fumagillin Control in Honey Bees. PLoS Pathog 9, e1003185 (2013).
35. Jack, C. J., Lucas, H. M., Webster, T. C. & Sagili, R. R. Colony Level Prevalence and Intensity of Nosema ceranae in Honey Bees (Apis mellifera L.). PLoS ONE 11, e0163522 (2016).
36. Johnston, B. A., Hooks, K. B., McKinstry, M. & Snow, J. W. Divergent forms of endoplasmic reticulum stress trigger a robust unfolded protein response in honey bees. J Insect Physiol 86, 1-10 (2016).
37. Keeling, P. J. & Fast, N. M. Microsporidia: biology and evolution of highly reduced intracellular parasites. Annu Rev Microbiol 56, 93-116 (2002).
38. Kent, M. L., Shaw, R. W. & Sanders, J. L. in Microsporidia: Pathogens of Opportunity (eds. Weiss, L. & Becnel, J. J.) (John Wiley and Sons, 2014).
39. Klee, J. et al. Widespread dispersal of the microsporidian Nosema ceranae, an emergent pathogen of the western honey bee, Apis mellifera. J Invertebr Pathol 96, 1-10 (2007).
40. Kupperman E, Lee E C, Cao Y, Bannerman B, Fitzgerald M, Berger A, et al. Evaluation of the Proteasome Inhibitor MLN9708 in Preclinical Models of Human Cancer. Cancer Research. 2010; 70: 1970-1980.
41. Lach, L., Kratz, M. & Baer, B. Parasitized honey bees are less likely to forage and carry less pollen. J Invertebr Pathol 130, 64-71 (2015).
42. Masood, M., Everett, C. P., Chan, S. Y. & Snow, J. W. Negligible uptake and transfer of diet-derived pollen microRNAs in adult honey bees. mabiology 13, 109-118 (2016).
43. Mayack, C. & Naug, D. Energetic stress in the honeybee Apis mellifera from Nosema ceranae infection. J Invertebr Pathol 100, 185-188 (2009).
44. Molestina, R., Becnel, J. J. and Weiss, L. M. 2014. Culture and propagation of microsporidia. pp. 457-467. In: Microsporidia—Pathogens of Opportunity (Weiss, L. M. and Becnel, J. J. eds.), Willey Blackwell, Oxford.
45. Nakjang, S. et al. Reduction and Expansion in Microsporidian Genome Evolution: New Insights from Comparative Genomics. Genome Biology and Evolution 5, 2285-2303 (2013).
46. Pombert J F, Selman M, Burki F. Gain and loss of multiple functionally related, horizontally transferred genes in the reduced genomes of two microsporidian parasites. 2012.
47. Potts, S. G. et al. Global pollinator declines: trends, impacts and drivers. Trends Ecol Evol (Amst) 25, 345-353 (2010).
48. Śledź P, Baumeister W. Structure-Driven Developments of 26S Proteasome Inhibitors. Annu Rev Pharmacol Toxicol. 2016; 56: 191-209.
49. SMITH, J. E. The ecology and evolution of microsporidian parasites. Parasitology 136, 1901-1914 (2009).
50. Stentiford, G. D. & Dunn, A. M. in Microsporidia: Pathogens of Opportunity (eds. Weiss, L. & Becnel, J. J.) 1-26 (John Wiley and Sons, 2014).
51. Stentiford, G. D. et al. Microsporidia-Emergent Pathogens in the Global Food Chain. Trends Parasitol (2016). doi: 10.1016/j.pt.2015.12.004
52. Szumowski, S. C. & Troemel, E. R. ScienceDirectMicrosporidia. Current Opinion in Microbiology 26, 10-16 (2015).
53. Texier, C., Vidau, C., Vigues, B., Alaoui, E I, H. & Delbac, F. Microsporidia: a model for minimal parasite-host interactions. Current Opinion in Microbiology 13, 443-449 (2010).
54. Traver, B. E. & Fell, R. D. Journal of Invertebrate Pathology. J Invertebr Pathol 107, 43-49 (2011).
55. Weiss L and Becnel J. Clinical Syndromes Associated with Microsporidiosis. Microsporidia: Pathogens of Opportunity. 2014.
56. Williams, G. R. et al. Standard methods for maintaining adult Apis mellifera in cages under in vitro laboratory conditions. Journal of Apicultural Research. 52, 1 (2013).
57. Wittner, M. & Weiss, L. The Microsporidia and Microsporidiosis. (American Society of Microbiology Press, 1999).

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation.

What is claimed is:

1. A method for treating an infectious disease in a subject in need thereof, comprising administering an effective amount of a proteasome inhibitor to the subject.

2. The method of claim 1, wherein the infectious disease is a microsporidia infection.

3. The method of claim 2, wherein the microsporidia infection is caused by Nosema ceranae.

4. The method of claim 1, wherein the subject is selected from the group consisting of insects, fish, birds and mammals.

5. The method of claim 1, wherein the subject is a honeybee.

6. The method of claim 1, wherein the proteasome inhibitor is selected from the group consisting of Bortezomib, Carfilzomib, Marizomib, Ixazomib, ixazomib citrate, Oprozomib, Delanzomib, MG132, Dexazomib, Epoxomicin, HMB-Val-Ser-Leu-VE, MG-262 and combinations thereof.

7. The method of claim 1, wherein the proteasome inhibitor is Ixazomib.

8. The method of claim 1, further comprising applying heat-shock to the subject.

9. A method for monitoring the progress of a microsporidia infection in a subject, comprising:
(a) obtaining a cell sample from the subject;
(b) staining the cell sample with one or more cell dyes;
(c) identifying distinct cell populations in the sample based on the dye signals;
(d) determining the infection stage of the subject by comparing the result of step (c) with that of an uninfected subject; and
(e) initiating a treatment protocol for the subject based on the infection stage determined in step (d).

10. The method of claim 9, wherein the microsporidia infection is caused by Nosema ceranae.

11. The method of claim 9, wherein the subject is a honeybee.

12. The method of claim 9, wherein the one or more cell dyes are selected from a chitin-binding dye, a lysosome dye, and combinations thereof.

13. The method of claim 9, wherein step (c) is carried out by flow cytometry.

14. A method for measuring the intensity of a microsporidia infection in a subject, comprising:
(a) obtaining a cell sample from the subject;
(b) measuring the infection intensity by determining the relative amount of the microsporidia DNA versus host DNA of the subject; and
(c) initiating a treatment protocol for the subject based on the infection intensity measured in step (b).

15. The method of claim 14, wherein the microsporidia infection is caused by Nosema ceranae.

16. The method of claim 14, wherein the subject is a honeybee.

17. The method of claim 14, wherein step (b) is carried out by quantitative PCR.

18. A method for treating a Nosema ceranae infection in a susceptible organism, comprising administering an effective amount of Ixazomib to the susceptible organism.

19. The method of claim 18, wherein the susceptible organism is a honeybee.

20. The method of claim 19, further comprising placing the honeybee in an environment with an elevated temperature of 45° C. for a sufficient time to treat the infection.
